(19) United States
(12) Patent Application Publication
(10) Pub. No.: US 2009/0048334 A1
(43) Pub. Date: Feb. 19, 2009

(54) Sodium ascorbate solubilisate

(76) Inventor: Dariush Behnam, Rossdorf (DE)

Correspondence Address: JACOBSON HOLMAN PLLC, 400 SEVENTH STREET N.W., SUITE 600, WASHINGTON, DC, 20004, US

(21) Appl. No.: 11/665,534

(22) PCT Filed: May 9, 2005
(86) PCT No.: PCT/EP05/04993

§ 371 Date: Apr. 17, 2007

(30) Foreign Application Priority Data
Oct. 20, 2004 (DE) 10 2004 051 245.0

(51) Int. Cl. A61K 31/355; A61K 31/375; A23L 1/302

(52) U.S. Cl. 514/458; 514/474; 426/72

(57) ABSTRACT

The invention relates to a solubilisate consisting of an aqueous solution of the alkali salts and/or alkaline-earth salts of ascorbic acid for foodstuffs, cosmetics and or similar and consisting of an emulsifier with an HLB value of between 9 and 18, for example a polysorbate, which also comprises one or more mid-chain triglycerides and/or one or more tocopherols.

In order to improve the life and stability of food, pharmaceuticals, cosmetics, and personal care products reduction agents are added to these products, which prevent their non-enzymatic oxidation as well as any undesired formation of aromas resulting therefrom. Ascorbic acid is widely used as a reduction agent, with its use being aggravated in that it is insufficiently stable in aqueous solutions. For example, in an aqueous ascorbic acid solution only approximately 70% of the added ascorbic acid is present after 30 days.

An increased stability of the reduction agent in aqueous solutions is achieved by derivation of the antioxidants. Here, for example, sodium ascorbic phosphate or ascorbylpalmitate are frequently used. Although the content of these agents remains constant in an aqueous solution over an extended period of time, however, the ascorbic acid derivatives are several times more expenses than pure ascorbic acid. Furthermore, in higher concentrations the ascorbic acid derivatives tend to crystallize and lead to a discoloration of the final product.

Additionally, the above-mentioned reduction agents show an insufficient effectiveness for preserving fatty substances due to their solubility in the fats and oils used in the above-mentioned products being too low. The reduction agent adheres to the exterior of the fat droplets and cannot develop its antioxidative effect on the interior of the fatty mixtures.

The document EP 1338271 A1 describes a fat and water soluble solubilisate with ascorbic acid, comprising polysorbate 80, which renders ascorbic acid fat-soluble. By adding a triglyceride the stability of the solubilisate is increased. Additionally, in this document the combined use of ascorbic acid and tocopherol-solubilisates is discussed, in which the mixed solubilisate is included in a portion of 3% ascorbic acid and 7% tocopherol. In the patent DE 19647352 C2 a water-soluble solubilisate of the fat-soluble reducer ubichinon Q₁₀ is described with the help of the solving agent polyoxyethylene-sorbitane monooleate (polysorbate 80), which can be vitaminized by the addition of vitamin E, which is also fat-soluble.

The invention is based on the object to provide a reduction agent in an easily mixable liquid, aqueous or fat-soluble form, which is suitable for preserving fatty foods. It shall be very stable over many months in high concentrations.

This object is attained according to the invention in a solubilisate, comprising an aqueous solution of alkali and/or earth-alkali salts of the ascorbic acid for foods, cosmetics, and the like and an emulsifier with an HLB-value from 9 to 18, for example polysorbate or cremophore. The alkali and/or earth alkali salts of ascorbic acid are particularly well suitable based on non-acidic features in an aqueous environment for attaining the above-mentioned object, particularly for such uses (e.g., ointments) in which a slightly acrid effect of the ascorbic acid is undesired. The solubilisate is clear and almost gelatinous at room temperature, can be thinned with water without becoming turbid and can easily be mixed, when heated to approximately 35° C. in aqueous as well as fatty end products of the cosmetics or food sector, primarily sausage or meat products of all sorts, without any additional processing steps. From professional literature the use of sodium-L-ascorbate in a crystalline form is known as an additive for the production of sausage and ham.

Therefore, preferably alkali and/or earth alkali salts of sodium-L-ascorbate are used, which are contained in the solubilisate, beneficially at a concentration from approximately 9% by weight to approximately 11% by weight. It is further beneficial to add one or more mid-chain triglycerides to the solubilisate at a concentration of preferably approximately 8% by weight to approximately 11% by weight, with the triglycerides beneficially containing octanoic acid and/or decanoic acid. The solubilisate may contain, instead of triglycerides, one or more vegetable oils, such as for example safflower oil, rape-seed oil, soy bean oil, or sunflower-seed oil in essentially identical percentages. Further, it has shown advantageous when the solubilisate contains one or more tocopherols or a tocopherol-mixture and/or derivatives therefrom, such as for example the acetate of the tocopherol, beneficially as a vitamin-E-acetate at a concentration from approximately 9% by weight to approximately 11% by weight.

The solubilisate according to the invention may contain a non-ionic emulsifier, properly licensed in the respective country for the food purpose according to the invention. Particularly beneficial are those with a HLB-value ranging from 9 to 18, because they lead to optically clear end products. Therefore, in many European countries a polysorbate may be used, preferably polysorbate 80 or polysorbate 20, having a HLB-value of 15, and a sufficient amount of hydrophilic and hydrophobic groups. The percentage of polysorbate in the solubilisate according to the invention ranges beneficially from approximately 50% by weight to approximately 70% by weight.

The production method according to the invention for the solubilisate mentioned is characterized in that the alkali and/or earth alkali salt of the ascorbic acid, beneficially sodium-L-ascorbate, is dissolved in warm water, preferably at a temperature from approximately 60° C. to approximately 85° C., with an emulsifier having a HLB-value ranging from 9 to 18 being added to the solution when warm and agitated until clear. It is beneficial to dissolve one or more mid-chain triglycerides in the warm solution, containing for example octanoic acid and/or decanoic acid, and then to add and agitate the emulsifier, beneficially a polysorbate such as, for example, polysorbate 80 or polysorbate 20. Instead of triglycerides tocopherols or derivatives therefrom, such as for example tocopherol acetate, may also be added to and agitated in the solution. Further, one or more vegetable oils may also be used instead of the mid-chain triglycerides, such as for example safflower oil, rape-seed oil, soy bean oil, or sunflower-seed oil.

The solubilisate according to the invention may also be used as an additive to foods, primarily meat and sausage products of all types, for preserving oils, fats, emulsions, as well as drugs.

It is well known that ascorbate oxidize relatively easily. Based on this characteristic, a further development of the invention improves the life of alkali and/or earth alkali salts by the solubilisate micelles each being additionally provided with one or more tocopherols or a tocopherol mixture. Each solubilisate micelle then contains both the ascorbate as well as the tocopherol and/or a mixture of individual ascorbate and tocopherol micelles.

For the above-mentioned solubilisates, for example, the alkali salt sodium-L-ascorbate can be used as the base, which primarily ensures the color stability of meat and sausage products. In the solubilisate α-tocopherol, tocopherol derivatives, and/or tocopherol-mixtures can be used as tocopherols.

The sodium-L-ascorbate content of the formulation according to the invention, which may amount up to 11% by weight, remains practically free from loss over months. The vitamin E-acetate content in the solubilisate amounts to approximately 10% by weight. The polysorbate content beneficially amounts to approximately 69% by weight.

The sodium-L-ascorbate/vitamin E-acetate-solubilisate according to the invention can easily be added to foods, drugs, cosmetics (skin and hair care products) with the consequence that the integrity of these products is considerably increased. The polysorbate content of the solution according to the invention develops a type of retard function for the anti-oxidative features of ascorbic acid as well as the tocopherol, if applicable, for the easily oxidating contents of pharmaceutical formulations and foods. This way the desired protective function is maintained over a considerably longer period of time.

A method for producing the solubilisate according to the invention provides that alkali and/or earth alkali salts of the ascorbic acid are dissolved in warm water and an emulsifier with a HLB-value ranging from 9 to 18 is added while heated, preferably a polysorbate, and agitated until clear. It is useful to add the emulsifier polysorbate 20 and/or polysorbate 80 at a temperature of approximately 70 to 75° C., and to heat the mixture under agitation for some minutes until clear and homogenous. Spherical micelles develop when the emulsifier is added.

In a further development of the invention tocopherol oil is added to the aqueous solution of alkali and/or earth alkali salts of the ascorbic acid at a temperature of approximately 85° C., which leads to the formation of an emulsion. Subsequently the emulsifier polysorbate 20 is added at a temperature of approximately 85° C. The mixture is held for some minutes under agitation at the temperature listed until clear and homogenous. Spherical micelles develop when the emulsifier is added, in which both the ascorbate as well as vitamin E molecules are enclosed. Instead of tocopherol or additionally thereto an addition of mid-chain triglycerides may also be considered, such as liquid vegetable oil.

While the polysorbate molecules, at the beginning of the micelle formation, attach at the surface, the vitamin E-molecules associate to the hydrophobic areas of the emulsifier molecules. After exceeding the critical concentration of the micelle formation spherical micelles begin to form from the aggregate surfaces, with an aqueous compartment with dissolved alkali and/or earth alkali salts of the ascorbic acid being enclosed in their interior. In an aqueous environment the micelles are structurally provided with a double-walled shell of radially arranged polysorbate molecules. Here, the polysorbate molecules of the interior shell enclose with their hydrophilic section the polysorbate molecules the interior compartment with the enclosed alkali and/or earth alkali salt solution of the ascorbic acid and the hydrophilic sections of the polysorbate molecules of the exterior shell align towards the exterior. The vitamin E is associated to the hydrophobic section of the polysorbate molecule. The anti-oxidants are therefore micelles together in the solubilisate. Due to the fact that the reduction agents enclosed in the micelles are released only with a delay the anti-oxidative effect of the solubilisate remains effective in the agents mentioned for a longer period of time than, for example, when added alone to the ascorbylpalmitate, thus one of the ascorbic acid derivatives mentioned at the outset. Furthermore, the inclusion of the vitamin E, insoluble in water, in the micelle causes a distinct stabilization of the ascorbic acid, very easily decomposable as an enediol. By the joint implementation of the two reducers in the micelle the ascorbic acid is additionally protected from disintegration, leading to an increased stability thereof.

With regard to the use of the solubilisate according to the invention it is very advantageous that the water-soluble sodium ascorbate and the fat-soluble vitamin E are provided in the mixed micelles both in a water as well as a fat soluble form.

In order to allow a longer shelf life of the formulation the addition of one or more mid-chain triglycerides or vegetable oils to the solubilisate is advantageous, such as for example safflower oil, its concentration should approximately amount from 10 to 11% by weight, which reliably prevents the precipitation of parts of the reduction agent.

In food the inclusion of anti-oxidative effective substances in micelles prevents by the emulsifier that in oral application of the solubilisate according to the invention the ascorbic acid and/or the vitamin E already develops its effect already in the middle of the intestinal tract, i.e. in the stomach and the duodenum, and is hereby consumed. Rather, the above-mentioned micelled effective agents are rather not absorbed until they reach the small intestines (without the participation of bile acid, bile salts, and enzymes several times better in quality.)

The following examples illustrate the invention.

### 1. EXAMPLE FOR A TEN-PERCENT CONCENTRATED SODIUM ASCORBATE SOLUBILISATE WITH TRIGLYCERIDES

Material:
1.) 10 g sodium-L-ascorbate of the company SYNOPHARM GmbH, Germany, article no. 700252, powdered
2.) 25 g distilled water
3.) 55 g polysorbate 20 of the company Cognis, U.S.A., article no. 638252
4.) 10 g mid-chain triglycerides (miglyol 812) or vegetable oil

Method:
Approximately 10 g sodium-L-ascorbate is dissolved until clear with approximately 25 g distilled water at approximately 70 to 75° C. Subsequently 55 g polysorbate 20 is added to this mixture and agitated at the indicated temperature until the mixture has reached transparency and homogeneity. Approximately 10 g of mid-chain triglycerides is added to the solution under agitation at a temperature below 50° C. and agitated until clear and transparent.

### 2. EXAMPLE FOR A NINE-PERCENT CONCENTRATED SODIUM ASCORBATE SOLUBILISATE WITH TRIGLYCERIDES

Material:
1.) 9 g sodium-L-ascorbate of the company SYNOPHARM GmbH, Germany, article no. 700252, powdered
2.) 24 g distilled water
3.) 56 g polysorbate 20 of the company Cognis, U.S.A., article no. 638252
4.) 11 g mid-chain triglycerides (miglyol 812) or vegetable oil

Method:
Similar to example 1, 9 g sodium-L-ascorbate is dissolved in approximately 24 g distilled water and approximately 56 g polysorbate 20 is added to this solution. After being heated to approximately 70 to 75° C. the mixture is agitated until it has reached transparency and homogeneity. Approximately 11 g of mid-chain glycerides is added to the solution at a temperature below 50° C. and agitated until clear and transparent.

### 3. EXAMPLE FOR A TEN-PERCENT CONCENTRATED SODIUM ASCORBATE SOLUBILISATE WITH TRIGLYCERIDES

Material:
1.) 10 g sodium-L-ascorbate of the company SYNOPHARM GmbH, Germany, article no. 700252, powdered
2.) 24 g distilled water
3.) 55 g polysorbate 80
4.) 11 g mid-chain triglycerides or vegetable oil

Method:
Similar to example 1, approximately 10 g sodium-L-ascorbate is dissolved in approximately 24 g of distilled water and adds approximately 55 g of polysorbate 80 is added to this solution. After being heated to approximately 70 to 75° C. this mixture is agitated until it has reached transparency and homogeneity. Approximately 11 g of mid-chain glycerides is added to the solution at a temperature below 50° C. and agitated until clear and transparent.

### 4. EXAMPLE FOR A TEN-PERCENT CONCENTRATED SODIUM ASCORBATE/VITAMIN E-ACETATE SOLUBILISATE

Material:
1.) 105.8 g sodium-L-ascorbate of the company SYNOPHARM GmbH, Germany, article number 700252, powdered
2.) 115 distilled water
3.) 94.7 g vitamin E-acetate of BASF AG, Germany, article number 50045159
4.) 684.5 g polysorbate 20 of the company Cognis, U.S.A., article number 638252

Method:
Approximately 105.8 g sodium-L-ascorbate is added to approximately 115 g water, heated to 85° C., and agitated and dissolved until clear at a constant temperature amounting to approximately 85° C. Subsequently, approximately 94.7 g of vitamin E-acetate is worked into the solution at a temperature of approximately 85° C. and the temperature is kept constant over the entire solution process. Then approximately 684.5 g polysorbate 20 is added to this mixture and agitated at 85° C. until the mixture reaches transparency and homogeneity. The solubilisate yielded is allowed to cool slowly and is bottled at approximately 60° C.

### 5. EXAMPLE FOR A TEN-PERCENT CONCENTRATED SODIUM ASCORBATE/MIXED TOCOPHEROL SOLUBILISATE

Material:
1.) 105.8 g sodium-L-ascorbate of the company SYNOPHARM GmbH, Germany, article number 700252, powdered
2.) 115 g distilled water
3.) 94.7 g tocopherol mixture of the company Archer-Daniels-Midland, U.S.A., (DECANOX MTS 90 MIXED TOCOPHEROLS), comprising tocopherols up to 90% by weight, composed as followed: 10% α-tocopherol, 1β-tocopherol, 59% γ-tocopherol, and 20% δ-tocopherol.
Or 94.7 g mixed tocopherol of the company COGNIS, U.S.A., (Covi-ox® T-90), comprising tocopherols up to 90% by weight, composed as follows: 14% α-tocopherol, 2% β-tocopherol, 60% γ-tocopherol, and 24% δ-tocopherol.
4.) 684.5 g polysorbate 20 of the company Cognis, U.S.A., article number 638252

Method:
Approximately 105.8 g sodium-L-ascorbate is added to approximately 115 g water, heated to approximately 85° C., and agitated to dissolve until clear at a constant temperature of approximately 85° C. Subsequently, approximately 94.7 g of the mixed tocopherol of the company Archer-Daniels-Midland Company, U.S.A. and/or the company Cognis, U.S.A. is worked into the solution at a temperature of approximately 85° C., and the temperature is kept constant over the entire dissolving process. Then approximately 684.5 g polysorbate 20 is added to this mixture and agitated at 85° C. until the mixture has reached transparency and homogeneity. The solubilisate yielded is allowed to slowly cool and it is bottled at approximately 60° C.

### 6. EXAMPLE FOR TEN-PERCENT CONCENTRATED SODIUM ASCORBATE-SOLUBILISATE WITH TRIGLYCERIDES

Material:
1.) 10 g sodium-L-ascorbate of the company SYNOPHARM GmbH, Germany, article number 700252, powdered
2.) 25 g distilled water
3.) 55 g polysorbate 80
4.) 10 g mid-chain triglycerides (miglyol 812) or vegetable oil

Method:
Similar to example 1, approximately 10 g sodium-L-ascorbate is dissolved in approximately 25 g distilled water and approximately 55 g polysorbate 80 is added to this solution. After warming to approximately 70 to 75° C. this mixture is agitated until the mixture has reached transparency and homogeneity. Under agitation, approximately 10 g mid-chain glycerides are added to the solution at a temperature below 50° C. and agitated until clear and transparent.

### 7. EXAMPLE FOR A NINE-PERCENTAGE SODIUM ASCORBATE SOLUBILISATE

Material:
1.) 9.g sodium-L-ascorbate of the company SYNOPHARM GmbH, Germany, article number 700252, powdered
2.) 25 g distilled water
3.)8 g miglyol 812
4.) 58 g polysorbate 20 of the company Cognis, U.S.A., article number 638252

Method:
Approximately 9 g sodium-L-ascorbate is added to approximately 25 g water, heated to approximately 85° C., and agitated to dissolve until clear at a constant temperature of approximately 85° C. Subsequently approximately 8 g miglyol 812 is worked into the solution at a temperature of approximately 85° C. and the temperature is maintained constant over the entire solution process. Subsequently 58 g polysorbate 20 are added to this mixture and agitated at approximately 85° C. until the mixture has reached transparency and homogeneity. The solubilisate is slowly allowed to cool and bottled at approximately 60° C.
The production method may also be designed such, though, that the sodium-L-ascorbate is dissolved to clarity in water of 50° C. to 60° C. and subsequently the polysorbate 20 is added and the mixture is well homogenized. Here, the mixture is heated to approximately 75° C. to approximately 85° C. so that a clear solubilisate develops. After cooling to at least 50° C., the triglycerides are added under agitation. The temperature may not exceed 50° C., here.

### 8. EXAMPLE FOR A NINE-PERCENT CONCENTRATED SODIUM ASCORBATE SOLUBILISATE

Material:
1.) 9 g sodium-L-ascorbate of the company SYNOPHARM GmbH, Germany, article number 700252, powdered
2.) 25 g distilled water
3.) 66 g polysorbate 20 of the company Cognis, U.S.A., article number 638252

Method:
Approximately 9 g sodium-L-ascorbate is added to approximately 25 g water, heated to approximately 85° C., and agitated to dissolve until clear at a constant temperature of approximately 85° C. Subsequently approximately 66 g polysorbate 20 is added to this solution and agitated at approximately 85° C. until the mixture has reached transparency and homogeneity. The solubilisate yielded is allowed to slowly cool and is bottled at approximately 60° C.

### 9. EXAMPLE FOR A NINE-PERCENT CONCENTRATED SODIUM ASCORBATE SOLUBILISATE

Material:
The same material is used as in example 8, with the difference that the polysorbate 20 is replaced by the same amount of polysorbate 80.

Method:
The production process is the same as in example 8.
The solubilisate according to examples 1 through 7 have an excellent and long shelf life. On the other hand, the stable solubilisates produced according to examples 8 and 9 are provided for the use as soon as possible within the scope of the purpose intended.

For the above-listed exemplary applications of the invention the qualitative statements concerning the material used are irrelevant, only the ratios of this data. For example, 5750 g water can be used as the original material and accordingly the 50-fold higher values of the materials mentioned can be added in order to yield the same result. Instead of polysorbates, within the scope of the invention, cremophores of the series RH 40, A 6, A 25, EL may also be used, with their HLB-values ranging from 10 to 17.

1. A solubilisate comprising an aqueous solution of alkali and/or earth alkali salts of the ascorbic acid for foods, cosmetics, and the like as well as an emulsifier having a HLB-value of approximately 9 to approximately 18, for example polysorbate.

2. A solubilisate according to claim 1, in which the salts of the ascorbic acid comprise sodium-L-ascorbate.

3. A solubilisate according to claim 1, further comprising one or more mid-chain triglycerides.

4. A solubilisate according to claim 3, in which the triglycerides include octanoic acid and/or decanoic acid.

5. A solubilisate according to claim 1, further comprising one or more tocopherols or a mixed tocopherol and/or derivatives, such as acetates of tocopherol.

6. A solubilisate according to claim 1 having a content of sodium-L-ascorbate ranging from approximately 9% by weight to approximately 11% by weight.

7. A solubilisate according to claim 1 having a content of polysorbate of approximately 50% by weight to approximately 70% by weight.

8. A solubilisate according to claim 1 comprising polysorbate 80 or polysorbate 20.

9. (A solubilisate according to claim 5 with a content of triglycerides from approximately 8% by weight to approximately 11% by weight.

10. A solubilisate according to claim 1, in which the triglycerides are replaced by one or more vegetable oils, such as safflower oil, rape seed oil, soy bean oil, or sunflower seed oil.

11. A solubilisate according to claim 5, further comprising approximately 9% by weight to approximately 10% by weight vitamin E-acetate.

12. A solubilisate according to claim 11, in which the vitamin E-acetate is replaced by a mixed tocopherol.

13. A solubilisate according to claim 1 with a content of sodium-L-ascorbate of approximately 9 to approximately 10% by weight, a content of polysorbate of approximately 55 to approximately 56% by weight, and a content of triglyceride of approximately 8 to 11% by weight.

14. A solubilisate according to claim 1 with a content of water ranging from 10 to approximately 25% by weight.

15. A solubilisate according to claim 1 having a sodium-L-ascorbine content of approximately 11% by weight, vitamin E-acetate of approximately 9.5% by weight, and a content of polysorbate 20 of approximately 68.5% by weight.

16. A method for producing a solubilisate according to claim 1, in which the alkali and/or earth alkali salts of ascorbic acid are dissolved in warm water, an emulsifier, preferably a polysorbate having a HLB-value of 9-18, is added under heat and agitated until clear.

17. A method for producing a solubilisate according to claim 16, in which the alkali and/or earth alkali salt of the ascorbic acid is dissolved in warm water, tocopherol or tocopherol derivatives are added, and agitated into a homogenous emulsion, and a polysorbate is added under heat and agitated until clear.

18. A method according to claim 16, in which polysorbate 20 or polysorbate 80 is used.

19. A method according to claim 16, in which one or more mid-chain triglycerides or vegetable oils are added to the aqueous alkali and/or earth alkali salt solution of the ascorbic acid.

20. A method according to claim 16, in which the solution is prepared at a temperature from approximately 60° C. to approximately 85° C. and at this temperature the agitation with the emulsifier occurs.

21. A method according to claim 19, in which instead of triglycerides one or more vegetable oils, such as safflower oil, rape seed oil, soy bean oil, or sunflower seed oil is used.

22. The use of a solubilisate according to claim 1 as an addition to skin or hair care products, foods, primarily in meat and sausage products of all sorts, for the preservation of oils, fats, and emulsions as well as drugs.
